(19) United States
(12) Patent Application Publication
(10) Pub. No.: US 2015/0088023 A1
(43) Pub. Date: Mar. 26, 2015

(54) DEMOCRATIC NEUROTECHNOLOGY APPARATUS AND METHOD

(71) Applicant: INDONESIAN SCIENTIFIC SOLUTION CENTRE FOUNDATION (IS2C), Kudus (ID)

(72) Inventors: J. Dwidjaja Setiabudi, Kudus (ID); Kho Sasih Adisusanto, Kudus (ID); Setiawan Yusran, Kudus (ID); Max Arif Pramono, Kudus (ID); Robin Wijaya, Kudus (ID); Damar Suryawan, Kudus (ID); Eko Sulistiyono, Kudus (ID); PKA Budi Widianarko, Semarang (ID); Aloysius Lukas Soenarjo Soesilo, Salatiga (ID); Njoman Agung, Kudus (ID); Soenawan Tanoedjaja, Kediri (ID); Budi Hartanto, Kudus (ID); Eva Christin, Kudus (ID)

(73) Assignee: INDONESIAN SCIENTIFIC SOLUTION CENTRE FOUNDATION (IS2C), Kudus (ID)

(21) Appl. No.: 14/037,127

(22) Filed: Sep. 25, 2013

(51) Int. Cl. A61B 5/00; A61B 5/0476

(52) U.S. Cl. CPC A61B 5/4064; A61B 5/0476
USPC 600/544

(57) ABSTRACT

A method for monitoring at least one informative brainwave in at least one individual involved in forming a consensus about a subject to detect whether the at least one individual has formed the consensus in a responsible and fear-free manner comprising: detecting at least one informative brainwave in at least one individual involved in forming a consensus about a subject; processing data from the at least one informative brainwave in at least one individual involved in forming the consensus using a democratic neurotechnology algorithm for analyzing; and comparing input data with reference data to detect whether the at least one individual has formed the consensus in a responsible and fear-free manner; and storing the input and output data. A device for use in this method is also disclosed.

### FIELD OF THE INVENTION

This invention uses neurotechnology to monitor decision making in forums where consensus is to be reached by individuals to determine whether the decision making is responsible and fear free.

### BACKGROUND OF THE INVENTION

Neurotechnology is commonly understood to be any technology that has a fundamental influence on how people understand the brain and various aspects of consciousness, thought, and higher order activities in the brain. It also includes technologies that are designed to improve and repair brain function and allow researchers and clinicians to visualize the brain.

A decision-making process in a forum can be conducted through polling of opinions and discussion leading to a common agreement or consensus. This process is used by taking every individual's needs into consideration. In the process of decision making, every individual involved has to be free from fear so that the outcome is achieved in a responsible manner and is congruent with the conscience of each participant. In the field of neuropsychology this kind of skill is called emotion regulation (soft skill).

In this context, Pancasila can be put forward as an outstanding example of the main matrix of supreme guiding orientations every citizen or institution in Indonesia, or for the whole nation. Polling of opinion or decision making in any forum should manifest the core of the fourth principle of the Pancasila, that is, “Kerakyatan yang dipimpin oleh hikmat kebijaksanaan dalam permusyawaratan/perwakilan” (democracy).

The Democratic Neurotechnology Apparatus (DNA), wireless or non-wireless, is a beneficial instrument for monitoring and guiding an individual or a forum as they are engaged in a decision-making activity that is consistent with their conscience. The method of monitoring and guiding uses DNA comparison Matrix with individual informative brainwave. Informative brainwave is the inner self information which carried by theta, alpha and beta wave of everyone. The detection of informative brainwave uses a wireless or non-wireless electrode. The DNA method measures and analyzes the frequency, phase, time delay (tow theory) and amplitude of the informative brainwave of everyone. The DNA Matrix is the matrix of human being versus culture, attitude and behavior of everyone data base stored in memory.

The expression of human emotion can be detected through facial graphics, observing and detecting the neural activities surrounding the areas of the eyes, eyebrows, and the mouth, as has been indicated in a previous invention by Brush II, in the U.S. Pat. No. 5,732,232.

Farwell, in U.S. Pat. No. 5,406,956, entitled “Method and apparatus for truth detection,” discovered a method of detecting information stored in the brain of a subject, represented by several oddball series Probe, Targets and Irrelevant stimuli.

However, both Brush II and Farwell, in detecting truth, used stimulus questions which had been prepared beforehand, so the individuals could made the preparation before they took the test. Even for a psychopath, who is an expert in manipulating psychology, can make a good preparation and fake the test. In addition, each of the two inventions cannot be used to detect automatically, in the sense that there should be an observer present to observe changes in facial expressions or in brain waves by asking specific questions that had been prepared.

This invention of DNA is able not only to detect conscience-based polling, but also to serve as an instrument for inner self reflection, either individually or collectively. In a forum of creativity, this invention can serve as a guide that helps us in conducting “3is” (Invention, Innovation, and Improvement) to obtain a more optimal outcome.

### SUMMARY OF THE INVENTION

Described herein is a method for monitoring at least one informative brainwave in at least one individual involved in forming a consensus about a subject to detect whether the at least one individual has formed the consensus in a responsible and fear-free manner comprising:
detecting at least one informative brainwave in at least one individual involved in forming a consensus about a subject;
processing data from the at least one informative brainwave in at least one individual involved in forming the consensus using a democratic neurotechnology algorithm for analyzing and comparing input data with reference data to detect whether the at least one individual has formed the consensus in a responsible and fear-free manner; and
storing the input and output data.

The detecting of the at least one informative brainwave may be performed wirelessly or non-wirelessly.

In the method, the informative brainwave may be selected from at least one of the theta, alpha and beta brainwaves.

Also disclosed is a device for monitoring at least one informative brainwave in at least one individual involved in forming a consensus about a subject to detect whether the at least one individual has formed the consensus in a responsible and fear-free manner comprising:
at least one wireless detector of informative brainwaves for monitoring the at least one informative brainwave in the at least one individual ,
a processor using a democratic neurotechnology algorithm for analyzing and comparing input data with reference data to detect whether the at least one individual has formed the consensus in a responsible and fear-free manner; and
memory storage for input and output data.

Generally a number of individuals will be monitored as they work towards consensus on a subject or an issue in any type of forum. The monitoring may involve the collection of opinions as well as the monitoring of the at least one brainwave in all individuals.

The DNA method measures and analyzes the frequency, phase, time delay (tow theory) and amplitude of the at least one informative brainwave of each individual. The DNA Matrix is the matrix of human being versus culture, attitude and behavior of everyone's data base stored in memory.

### BRIEF DESCRIPTION OF THE DRAWINGS

Further features and advantages of the present invention will become apparent from the following detailed description, taken in combination with the appended drawings, in which:

FIG. 1 is a schematic illustration of one form of the present method; and

FIG. 2 is a schematic illustration of an algorithm of one embodiment of the present device.

### DETAILED DESCRIPTION OF THE PREFERRED EMBODIMENTS

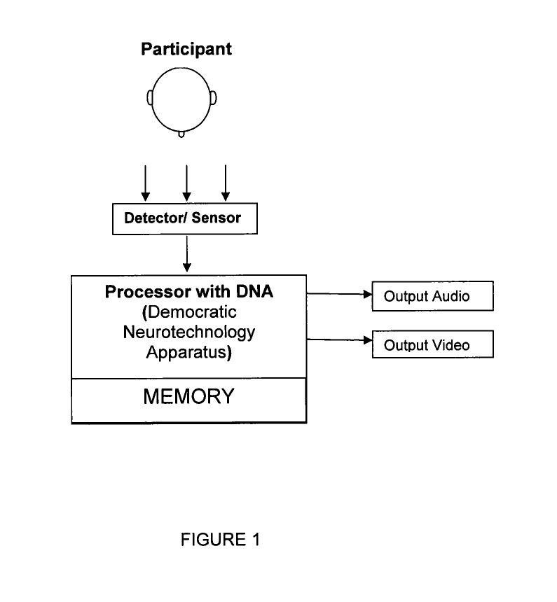

In reference to FIG. 1, the Detector/Sensor detects the informative brainwaves of a participant in a forum by means of wireless or non-wireless. The Processor with the DNA algorithm will analyze the informative brainwaves. The input and output data will be stored in memory. The outcome is in the form of an audio and/or video output.

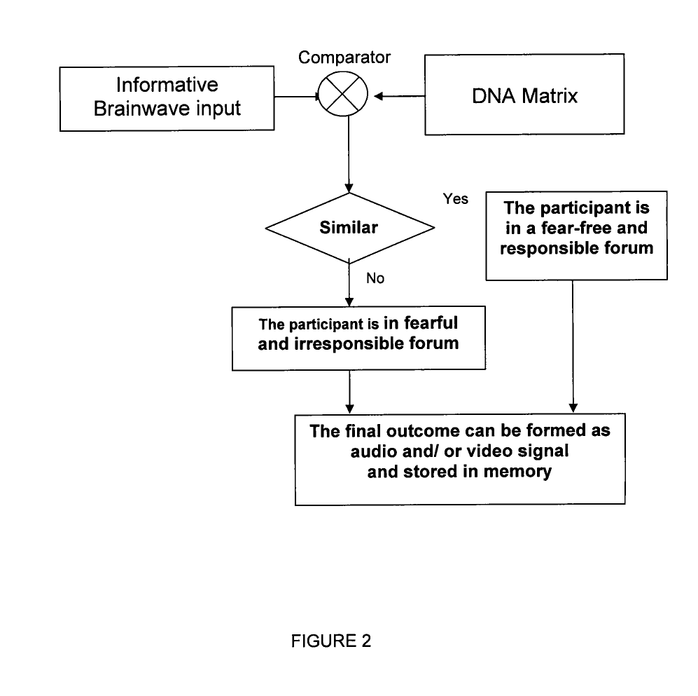

In reference to FIG. 2, the processor will compare the outcome of the informative brainwaves with the DNA matrix. If the participant is in a fear-free and responsible forum, then the result will be similar, and consistent with their consciences. On the other hand, in fearful and irresponsible forum, the result will be dissimilar, inconsistent with their conscience. The final outcome can be formed as an audio or video signal and stored in memory.

Certain adaptations and modifications of the described embodiments can be made. Therefore, the above discussed embodiments are considered to be illustrative and not restrictive.

1. A method for monitoring at least one informative brainwave in at least one individual involved in forming a consensus about a subject to detect whether the at least one individual has formed the consensus in a responsible and fear-free manner comprising:
detecting at least one informative brainwave in at least one individual involved in forming a consensus about a subject;
processing data from the at least one informative brainwave in at least one individual involved in forming the consensus using a democratic neurotechnology algorithm for analyzing and comparing input data with reference data to detect whether the at least one individual has formed the consensus in a responsible and fear-free manner; and
storing the input and output data.

2. A method as claimed in claim 1, wherein the detecting of the at least one informative brainwave is performed non-wirelessly.

3. A method as claimed in claim 1, wherein the detecting of the at least one informative brainwave is performed wirelessly.

4. A method as claimed in claim 1, wherein the informative brainwave is selected from at least one of the theta, alpha and beta brainwaves.

5. A device for monitoring at least one informative brainwave in at least one individual involved in forming a consensus about a subject to detect whether the at least one individual has formed the consensus in a responsible and fear-free manner comprising:
at least one wireless detector of informative brainwaves for monitoring the at least one informative brainwave in the at least one individual ,
a processor using a democratic neurotechnology algorithm for analyzing and comparing input data with reference data to detect whether the at least one individual has formed the consensus in a responsible and fear-free manner; and
memory storage for input and output data.

6. A device as claimed in claim 4, wherein the detecting of the at least one informative brainwave is performed with a non-wireless device.

7. A device as claimed in claim 4, wherein the detecting of the at least one informative brainwave is performed with a wireless device.

8. A device as claimed in claim 1, wherein the informative brainwave is selected from at least one of the theta, alpha and beta brainwaves.
